(19) United States
(12) Patent Application Publication
(10) Pub. No.: US 2023/0084613 A1
(43) Pub. Date: Mar. 16, 2023

(54) NON-INVASIVE BLOOD GLUCOSE SENSOR

(71) Applicant: Young Kwon PARK, Gyeonggi-do (KR)

(72) Inventor: Young Kwon PARK, Gyeonggi-do (KR)

(21) Appl. No.: 17/798,071

(22) PCT Filed: Dec. 29, 2020
(86) PCT No.: PCT/KR2020/019246

§ 371 Date: Aug. 6, 2022

(30) Foreign Application Priority Data
Feb. 18, 2020 (KR) 10-2020-0019525
Feb. 18, 2020 (KR) 10-2020-0026344

(51) Int. Cl. A61B 5/145; A61B 5/1477

(52) U.S. Cl. CPC A61B 5/14532; A61B 5/1477

(57) ABSTRACT

In general, regardless of using an optical method or an electrical AC resistance impedance method, non-invasive blood glucose measurement has a problem in that variations in the level of glucose contained in blood are too small to be measured as a signal, and thus measurement results are inaccurate due to noises generated during measurement and errors caused by the difficulty in consistent measurement. To solve this problem, the present invention provides a non-invasive blood glucose sensor includes: a measurement-unit body; an impedance electrode sensor provided on an inner bottom surface of the measurement-unit body; a signal-generation and measurement unit configured to measure impedance while scanning frequency by supplying multiple frequencies to the impedance electrode sensor; a blood sensor configured to measure the amount of blood flowing through a body part brought into contact with the impedance electrode sensor; and a status display LED configured to display different colors according to the amount of blood measured using the blood sensor. Owing to this configuration, the present invention has an effect of measuring an accurate blood glucose level in a non-invasive manner.

### TECHNICAL FIELD

The present invention relates to a method of measuring a blood glucose level using a non-invasive blood glucose sensor without taking a blood sample. More particularly, the present invention relates to a non-invasive blood glucose sensor configured to check the amount of blood glucose without having to take a blood sample by an invasive method, and a technique for improving the measurement accuracy of the non-invasive blood glucose sensor.

### BACKGROUND ART

Techniques prior to the application of the present invention involve: a step of measuring the impedance of a tissue fluid leaking through to the skin surface by using a blood glucose measurement unit of a non-invasive blood glucose sensor having a momentum measuring function, and then measuring the blood glucose level of the tissue fluid from the measured impedance; a step of measuring the momentum of a user using a momentum measurement unit of the non-invasive blood glucose sensor having the momentum measuring function and then calculating blood glucose variations according to the measured momentum; and a step of calculating an expected blood glucose level by comparing the calculated blood glucose variations with the measured blood glucose level, wherein the non-invasive blood glucose sensor having the momentum measuring function includes: the blood glucose measurement unit configured to take a tissue fluid through a user's skin surface and measure the impedance of the tissue fluid; the momentum measurement unit configured to measure the momentum of a user; and a processing unit configured to measure and calculate a blood glucose level, momentum, blood glucose variations, and an expected blood glucose level.

In addition, another prior technique relates to a non-invasive blood glucose sensor and a blood glucose measurement method. The prior technique provides a sufficiently accurate method for replacing the existing blood sampling method used by patients to measure their blood glucose levels, and a portable device using the sufficiently accurate method, wherein measured reflected light signals are appropriately filtered and mathematically processed to calculate a current blood glucose level.

### DISCLOSURE

### Technical Problem

In general, regardless of using an optical method or an electrical AC resistance impedance method, non-invasive blood glucose measurement has a problem in that variations in the level of glucose contained in blood are too small to be measured as a signal, and thus measurement results are inaccurate due to noises generated during measurement and errors caused by the difficulty in consistent measurement.

To solve the above-mentioned problem, the present invention provides a non-invasive blood glucose sensor including a signal generator and a signal measurement unit which have a frequency scanning function in which impedance is measured by generating multiple frequencies in order to use the feature in which blood glucose sensitivity varies with frequency bands, wherein data on the impedance of blood varying with frequency bands is measured, and the data is matched with blood glucose level variations.

In addition, when the blood glucose level of a person is repeatedly measured at the same time by a non-invasive method, there is a problem in that measurement results vary depending on factors such as the contact area of a measurement site, a contact pressure applied to the measurement site, and the dryness of a finger surface.

### Technical Solution

The present invention provides the following means in order to solve the above-described problems.

The present invention provides a non-invasive blood glucose sensor including: a measurement-unit body of the non-invasive blood glucose sensor;

an impedance electrode sensor provided on an inner bottom surface of the measurement-unit body;

a signal-generation and measurement unit configured to measure impedance while scanning frequency by supplying multiple frequencies to the impedance electrode sensor;

a pressure sensor configured to measure a contact pressure of a body part brought into contact with the impedance electrode sensor; and

a notification unit configured to indicate whether the contact pressure measured using the pressure sensor is appropriate.

In addition, a status display LED displays green when the contact pressure measured using the pressure sensor is appropriate for non-invasive blood glucose measurement, yellow when the contact pressure is insufficient, and red when the contact pressure is excessive.

In addition, the impedance electrode sensor may be connected in parallel to a coil and in series to a capacitor to form a sensor circuit.

In addition, the impedance electrode sensor may be connected in series to a capacitor connected in parallel to a coil to form a sensor circuit.

As another means for solving the above-described problems, the present invention provides a non-invasive blood glucose sensor including:

a measurement-unit body of the non-invasive blood glucose sensor;

an impedance electrode sensor provided on an inner bottom surface of the measurement-unit body;

a signal-generation and measurement unit configured to measure impedance while scanning frequency by supplying multiple frequencies to the impedance electrode sensor;

a blood sensor configured to measure an amount of blood flowing through a blood vessel so as to measure a magnitude of a contact signal of a body part brought into contact with the impedance electrode sensor; and

a notification unit configured to indicate whether the amount of blood measured using the blood sensor is appropriate.

In addition, the notification unit includes a status display LED configured to display green when the amount of blood measured using the blood sensor is appropriate for non-invasive blood glucose measurement, yellow when the amount of blood is insufficient, and red when the amount of blood is excessive.

In addition, the impedance electrode sensor may be connected in parallel to a coil and in series to a capacitor to form a sensor circuit.

In addition, the impedance electrode sensor may be connected in series to a capacitor connected in parallel to a coil to form a sensor circuit.

### Advantageous Effects

The present invention configured as described above has an effect of non-invasively measuring an accurate blood glucose level by constantly maintaining the contact area and pressure between a sensor and the body of a user who measures his/her blood glucose level, and sensing the surface state of a measurement site such as a finger for consistent measurement.

### BRIEF DESCRIPTION OF DRAWINGS

FIG. 1 is a conceptual view illustrating an impedance electrode sensor of the present invention.

FIG. 2 is a circuit diagram illustrating a peripheral circuit around the impedance electrode sensor of the present invention.

FIG. 3 shows impedance-frequency graphs obtained in the circuit condition shown in section (a) of FIG. 2 according to the present invention.

FIG. 4 shows impedance-frequency graphs obtained in the circuit condition shown in section (b) of FIG. 2 according to the present invention.

FIG. 5 is a perspective view illustrating a measurement-unit body and a status display LED according to the present invention.

FIG. 6 shows the measurement-unit body and the impedance electrode sensor according to the present invention.

FIG. 7 is a view illustrating the overall configuration of a non-invasive blood glucose sensor of the present invention.

FIG. 8 is a graph illustrating blood glucose impedance values measured by a conventional method.

FIG. 9 is a conceptual view illustrating a function M for calculating a blood glucose level according to the present invention.

### BEST MODE

### Mode for Invention

The development of science and medical technology has made it easier to cure diseases and increased the life expectancy of modern people, and thus the modern society has entered an aging society. Therefore, the interest of modern people has expanded to preventing diseases as well as curing diseases. The most typical example of modern diseases may be diabetes. Diabetes is a chronic disease causing numerous complications. Thus, once diabetes develops, consistent lifetime care is necessary. To this end, regular blood glucose level measurement is required.

In general, most blood glucose sensors are configured to directly measure a blood glucose level using a blood sample taken from the tip of a finger. A user pierces the skin with a lancet and then squeezes a finger until a significant amount of blood is drawn onto an appropriate test strip. The strip is placed in a glucometer that determines the concentration of glucose. Such invasive blood glucose sensors determine the concentration of glucose by using a reflected-light photometry method, an absorbance photometry method, or an electrochemical method depending on the types thereof. Since blood samples are obtained by directly piercing the skin in such methods, most young patients with long-standing diabetes complain of the inconvenience of “invasive” methods in which a blood sample is directly taken. In addition, invasive methods may cause infection.

A big issue in this field is the sensitivity for fine distinguishment at low concentrations ranging from 0 mg/dL to 1,000 mg/dL. Currently, much research is conducted into non-invasive glucose monitoring systems using saliva, tears, sweat, and the like. However, many non-invasive techniques still require expensive and complicated systems. Electromagnetic wave sensors having non-destructive characteristics for subjects to be measured have great potential in the medical field, and thus much research is currently conducted into electromagnetic wave sensors. To this end, the interaction between electromagnetic waves and living tissue is very important in terms of performance improvements. Most of such methods are to measure the concentration of glucose in blood in a completely non-invasive manner in the near field of electromagnetic radiation by using a relationship between glucose concentration and dielectric constant. In such a method, how deep electromagnetic waves penetrate into living tissue is an important design parameter for high sensitivity. Recently, studies on small planar resonators or radiators that may easily come into contact with the human body have also been reported as specific attempts to conveniently measure a blood glucose level in a non-invasive wireless manner.

However, most of these non-invasive blood glucose measurement methods are limited in improving sensitivity because of reflection near the skin or subcutaneous tissue, or excessive resonance frequency shifts. A resonator is placed on a site in which a blood vessel or muscle containing a large amount of blood is located so as to measure a blood glucose level. However, such tissue has a high dielectric constant and high conductivity as well, thereby causing high loss.

The present invention provides: a simple and economical non-invasive blood glucose sensor which uses a resonator sensor and a method of measuring impedance variations at multiple resonant frequencies of the resonant sensor; and a technique for mapping measured nonlinear impedance data to blood glucose values of a subject.

Hereinafter, effects of the present invention will be described with reference to the drawings.

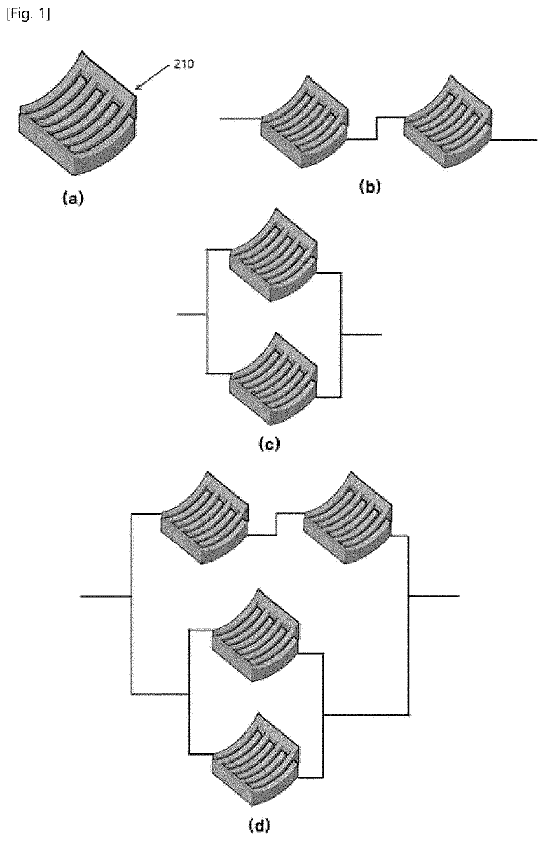

FIG. 1 shows an impedance electrode sensor having an interdigital electrode structure according to the present invention. Lines of the impedance electrode sensor have different polarities, thereby maximizing the directivity of an electromagnetic field going away from the lines. When a finger is placed on the lines, impedance varying with the blood glucose level of vessels in the skin is measured by causing an electromagnetic field to maximally penetrate into the finger. The total capacitance of the impedance electrode sensor (interdigital electrodes) may be adjusted by adjusting the thicknesses of interdigital electrodes and the distance between the interdigital electrodes. In addition, a circuit for generating magnetic resonance may be connected in parallel or series to the interdigital electrode structure such that magnetic resonance may occur at a desired frequency.

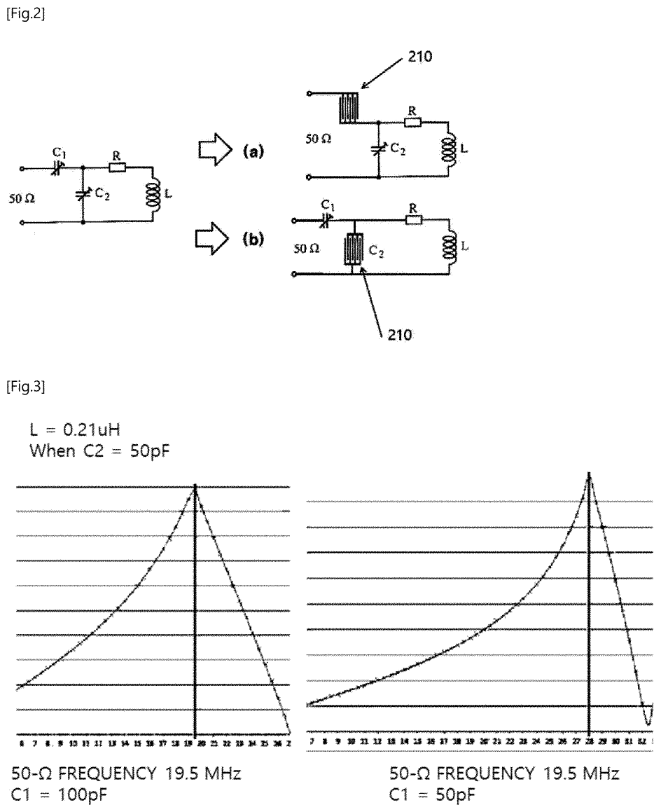

Section (a) of FIG. 1 shows a basic electrode structure, and sections (b), (c), and (d) of FIG. 1 show impedance electrode sensors connected in series (section (b)), in parallel (section (c)), and series-parallel (section (d)) according to the present invention so as to increase an electrode area and increase, using circuits shown in FIG. 2, measured impedance values varying with a blood glucose level. In addition, the size of each impedance electrode sensor may be varied.

The circuit on the left in FIG. 2 is a frequency tuning circuit, which is commonly called a tank circuit.

Here, a capacitor of the frequency turning circuit may be replaced with an impedance electrode sensor of the present invention. Left section (a) in FIG. 2 shows a circuit in which a capacitor C1 is replaced with an impedance electrode sensor of the present invention, and left section (b) in FIG. 2 shows a circuit in which a capacitor C2 is replaced with an impedance electrode sensor of the present invention. Ideally, the circuits are constructed by devices having no power loss. In reality, however, parasitic resistance, capacitance, and inductance components are everywhere in the circuits.

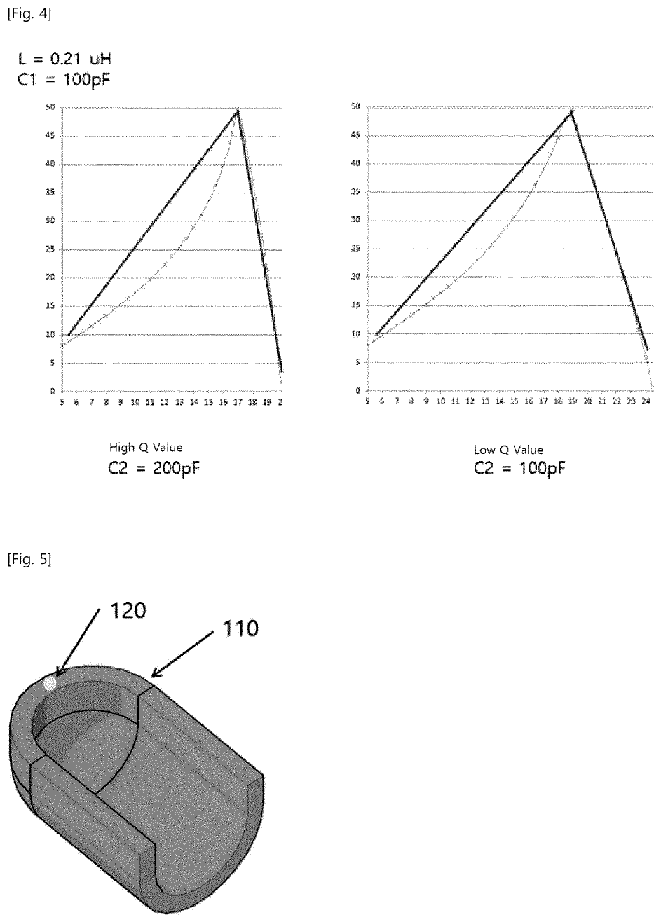

Operational principles of the circuits shown in FIG. 2 which are provided with the impedance electrode sensors shown in FIG. 1 will now be described with reference to FIGS. 3 and 4.

In operation, the impedance electrode sensor of the present invention shown in FIG. 1 functions as a capacitor in a circuit. An electromagnetic field is formed over the interdigital electrodes of the impedance electrode sensor, and the capacitance of the impedance electrode sensor varies depending on the permittivity of a material filled in the space of the electromagnetic field. Thus, the permittivity of the material may be measured by detecting a capacitance signal using a measurement unit connected to the circuit. When measuring a blood glucose level, an object placed on the impedance electrode sensor may be a finger or a body part, and since the permittivity of blood in the finger or the body part varies with blood glucose level variations, the permittivity of blood may be measured to measure a blood glucose level.

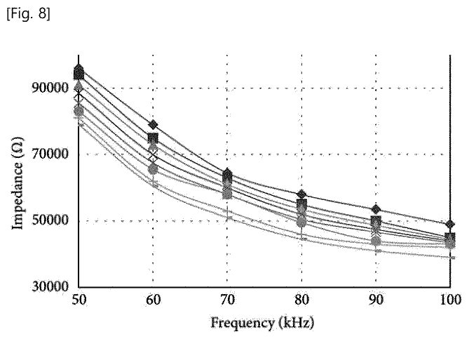

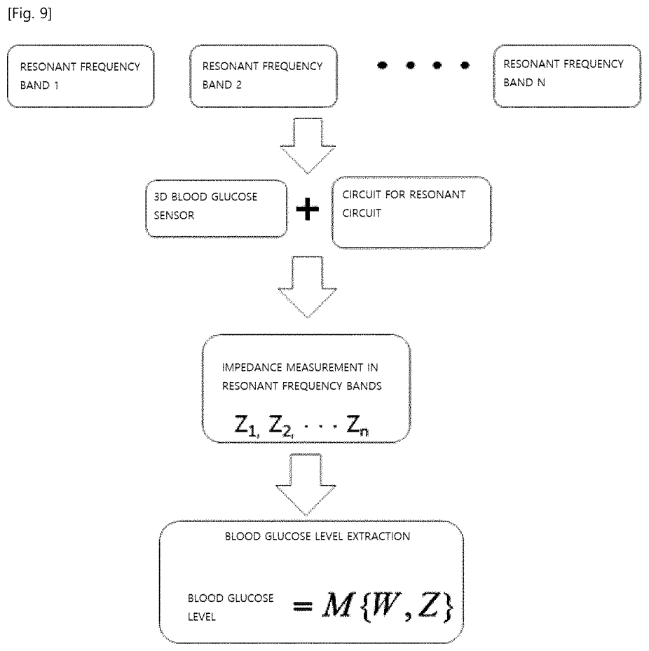

However, in conventional impedance measurement methods, the impedance of a sensor is directly measured without using a circuit such as those shown in FIG. 2, and thus only small differences are measurable according to impedance as shown in FIG. 8. Therefore, the conventional impedance measurement methods result in large errors according to external noises and measurement conditions. Here, factors such as the contact area, the contact pressure, and the moisture content of the contact surface between the impedance electrode sensor and the finger or the body part may act as external noises. That is, all factors that affect the measurement of permittivity except for blood glucose may be noises.

Therefore, the inventor intended to develop a technique for amplifying a blood glucose measurement signal and removing factors causing permittivity variations other than blood. To this end, it is intended to amplify and measure blood glucose level variations by using a circuit constructed by replacing a capacitor of the tuning circuit shown in FIG. 2 with the impedance electrode sensor.

The circuit shown in section (a) of FIG. 2 is constructed by replacing the capacitor connected in series to the tuning circuit with the impedance electrode sensor of the present invention. As shown in FIG. 3, in this circuit, a tuning frequency for matching an output impedance to 50Ω decreases as the capacitance of the impedance electrode sensor increases. Therefore, the feature, in which the tuning frequency decreases as the blood glucose level of a subject increases, may be compared with a method of measuring a blood glucose level by measuring the magnitude of a signal as shown in FIG. 8, so as to more accurately measure a blood glucose level.

The circuit shown in section (b) of FIG. 2 is constructed by replacing the capacitor connected in parallel to the tuning circuit with the impedance electrode sensor of the present invention. As shown in FIG. 4, in this circuit, a tuning frequency for matching to 50Ω varies little as the capacitance of the impedance electrode sensor increases. However, the Ω value of the tuning circuit increases as the capacitance of the impedance electrode sensor increases.

In order to take advantage of these relational variations, one impedance electrode sensor may be used as shown in FIG. 2, or two impedance electrode sensors may be used as shown in sections (a) and (b) of FIG. 2 to use all the relational variations at the same time.

A method of measuring impedance according to blood glucose level variations by using the non-invasive blood glucose sensor having the circuit shown in section (a) in FIG. 2 will now be described in detail.

Step A1: The impedance electrode sensor is brought into contact with a finger or a body part of a user or patient having a normal or reference blood glucose level.

In general, an index finger may be appropriate.

Step A2: In this case, a 50-Ω tuning frequency (or 50-Ω peak frequency) is measured using a frequency-scan and impedance-measurement circuit.

Step A3: The blood glucose level of the user or patient is measured using an accurate blood glucose measurement method, and the measured blood glucose level is set as a reference A1.

Step A4: After waiting one to three hours in a state in which the user or patient does not eat for variations in the blood glucose level of the user or patient, steps A1 to A3 are repeated to set a reference A2.

Step A5: A reference A3 is set by repeating steps A1 to A3 30 minutes after the user or patient has a meal.

Step A6: The blood glucose levels set as the references A1 to A3, and the 50-Ω tuning frequency (peak frequency) are input to the non-invasive blood glucose sensor of the present invention for use as a blood glucose measurement calibration curve.

The frequency-scan and impedance-measurement circuit has a frequency scanning range of 1 MHz to 100 MHz and may particularly perform frequency scanning within the range of 10 MHz to 50 MHz for fast scanning.

A method of measuring impedance according to blood glucose level variations by using the non-invasive blood glucose sensor having the circuit shown in section (b) in FIG. 2 will now be described in detail.

Step B1: The impedance electrode sensor is brought into contact with a finger or a body part of a user or patient having a normal or reference blood glucose level. In general, an index finger may be appropriate.

Step B2: In this case, a 50-Ω tuning frequency (or 50-Ω peak frequency) and at least one specific frequency within 2 MHz to 10 MHz to the left and/or right of the 50-Ω tuning frequency (or 50-Ω peak frequency) are set, and impedance values at the 50-Ω tuning frequency and the specific frequency are measured using the frequency-scan and impedance-measurement circuit.

Step B3: The blood glucose level of the user or patient is measured using an accurate blood glucose measurement method, and the measured blood glucose level is set as a reference B1.

Step B4: After waiting one to three hours in a state in which the user or patient does not eat for variations in the blood glucose level of the user or patient, steps B1 to B3 are repeated to set a reference B2.

Step B5: A reference A3 is set by repeating steps B1 to B3 30 minutes after the user or patient has a meal.

Step B6: The blood glucose levels set as the references B1 to B3, and impedance values at the 50-0 tuning frequency (peak frequency) and the set specific frequency are used to calculate Q values from tuning frequency variations according to blood glucose variations and the impedance value at the set specific frequency so as to use the Q values as a calibration curve for the non-invasive blood glucose sensor of the present invention.

The frequency-scan and impedance-measurement circuit has a frequency scanning range of 1 MHz to 100 MHz and may particularly perform frequency scanning within the range of 10 MHz to 50 MHz for fast scanning.

In addition, as described above, when two or more impedance electrode sensors are used, blood glucose levels corresponding to impedance values at a tuning frequency and a set frequency may be measured to prepare a calibration curve through deep learning, A1 learning, multiple regression analysis, or the like and may input the calibration curve to the non-invasive blood glucose sensor of the present invention.

In addition, the impedance electrode sensor may be used as a humidity measurement sensor that is not connected to the tuning circuit and used to individually measure the humidity on a skin surface. The degree of dryness of the skin surface may be detected by measuring only the impedance of the skin surface to determine whether an impedance value measured by the impedance electrode sensor reflects only variations in the permittivity of blood or also reflects variations in permittivity caused by the humidity on the skin surface. The impedance of the skin surface measured as described above may be used to correct results of blood impedance measurement, thereby improving blood glucose measurement precision. To this end, impedance measurement for measuring the humidity on a skin surface may be simply performed at a set frequency without frequency scanning. The set frequency for measuring the moisture of the skin may be low on the level of several hundred kilohertz (kHz), thereby guaranteeing a small skin penetration depth.

FIG. 5 relates to a method for securing precision during repetition of measurements, which is another problem to be solved by the present invention. Blood glucose is quantified by measuring the concentration of glucose in blood, and blood glucose measurement is affected by factors such as the contact pressure, area, and surface state between a body part of a subject and a measurement device. For blood glucose measurement, the present invention provides a means for maintaining consistent measurement conditions between a body part of a subject and a measurement device.

To this end, FIG. 5 shows a measurement-unit body provided with a notification unit such as a status display LED, which displays green when the pressure between a body part of a subject and a measurement device is appropriate for measurement, yellow when the pressure is insufficient, and red when the pressure is excessive.

The notification unit may include one or more of light, color, sound, vibration, and LCD units. The notification unit informs a user of an appropriate measurement pressure and may be any means capable of indicating whether a pressure applied by a body part is high or low. A light may be blinked at a high frequency to indicate a high pressure and at a low frequency to indicate a low pressure, and may be maintained in a turned-off or turned-on state to indicate an appropriate pressure. The notification unit using vibration may be used in the same method as described above. The LCD unit may display text, characters, or symbols.

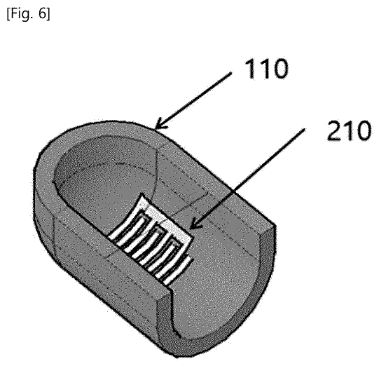

FIG. 6 is a view illustrating an impedance electrode sensor provided on a lower inner side of a measurement-unit body according to the present invention. The impedance electrode sensor may be provided only on a lower inner side of the measurement-unit body or may be provided on the entirety of the measurement-unit body. In addition, the impedance electrode sensor may be provided in one piece or several pieces as described above. That is, although FIG. 2 shows that one of the capacitors C1 and C2 is replaced with an impedance electrode sensor, it is also possible that both the capacitors C1 and C2 are replaced with impedance electrode sensors. A design, in which a plurality of impedance electrode sensors are connected in series or parallel to amplify impedance measured according to variations in blood glucose, may also be possible. Different calibration methods may be required according to such combinations, and to this end, methods such as multiple regression analysis, deep learning, backpropagation learning, or online learning may be used.

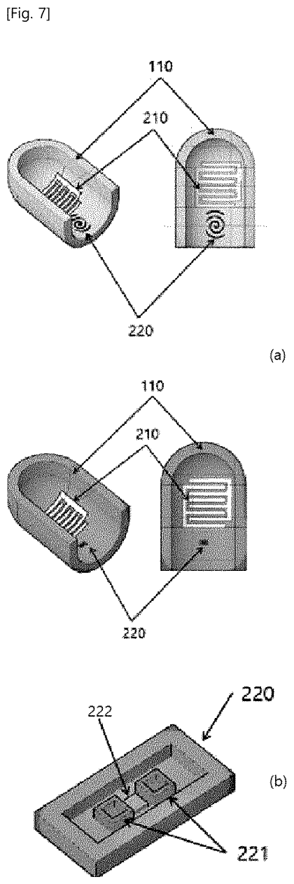

FIG. 7 shows a perspective view and a plan view illustrating an impedance electrode sensor and a pressure sensor according to the present invention. The pressure sensor may be provided on a part to be brought into contact with a measurement device to measure a contact pressure and maintaining the same contact pressure for every measurement. That is, a means for measuring blood glucose under the same measurement conditions is provided. In other words, blood glucose is quantified by measuring the concentration of glucose in blood, and results of measurement are affected by the pressure between the measurement device and a body part because the amount of blood in the body part varies according to the pressure between the measurement device and the body part. That is, when the pressure is excessively low, measurement is impossible, and when the pressure is excessively high, measurement is inaccurate because blood flows away from a measurement site due to the excessively high pressure. To solve this problem, the present invention is provided with the pressure sensor and a means for reading a measured value when an appropriate pressing pressure is applied.

Another embodiment using a blood sensor will now be described with reference to FIG. 7.

Section (a) of FIG. 7 shows a perspective view and a plan view illustrating an impedance electrode sensor and a blood sensor according to the present invention.

The blood sensor is provided in a contact portion between a user's body and a measurement device to measure the contact pressure between the user's body and the non-invasive blood glucose sensor of the present invention using the blood sensor capable of measuring the amount of blood, and thus the same contact pressure may be maintained for every measurement. That is, a means for measuring blood glucose under the same measurement conditions is provided. In other words, blood glucose is quantified by measuring the concentration of glucose in blood, and results of measurement are affected by the contact pressure between the measurement device and a body part because the amount of blood in the body part varies according to the contact pressure between the measurement device and the body part. That is, when the contact pressure is excessively low, measurement is impossible, and when the contact pressure is excessively high, measurement is inaccurate because blood flows away from a measurement site due to the excessively high pressure. To solve this problem, the present invention is provided with the blood sensor capable of measuring the amount of blood and a means for reading a measured value when an appropriate pressure is applied from a user's body to the non-invasive blood glucose sensor of the present invention. The blood sensor of the present invention shown in section (b) of FIG. 7 includes an infrared photosensor 222 between two infrared LEDs 221. When infrared light is emitted from the infrared LEDs toward a user's body, the infrared light propagates toward a blood vessel through which a large amount of blood passes. Then, the infrared photosensor detects infrared light reflected from the blood vessel.

The infrared photosensor mainly detects infrared light reflected from the blood vessel, and since the amount of blood in the blood vessel varies periodically according to the beating of the heart, measured values also vary according to variations in heartbeat. Therefore, since the amount of blood varies with the heartbeat, it is necessary to correct a measured impedance value according to the amount of blood in order to accurately measure a blood glucose level using an impedance method.

To this end, in the present invention, the magnitude of a signal measured by the infrared photosensor is detected and stored together with an impedance value and a tuning frequency which are measured with the frequency-scan and impedance-measurement circuit, so as to correct impedance variations according to the amount of blood.

In a method of correcting impedance variations according to the amount of blood, an increase in permittivity is calculated using the average of the maximum signal magnitude and the minimum signal magnitude which are measured with the infrared photosensor according to the heartbeat. However, when the maximum signal magnitude is excessively low, the status display LED turns red, and when the minimum signal magnitude is excessively low, the status display LED turns yellow. The status display LED turns green to inform a user that a measurement is possible. A user may adjust the contact pressure between a body part (usually a finger) of the user and the non-invasive blood glucose sensor of the present invention according to light emitted from the status display LED, and thus when measuring impedance, the amount of blood may be maintained constant for consistent measurement.

In another non-invasive blood glucose measurement method using a blood sensor, a blood glucose level is measured while scanning frequency at the moment when the signal of the blood sensor is maximal, so as for measurement with a large signal by measuring impedance when the amount of blood in a finger is maximal. In this case, the absolute signal magnitude of the blood sensor is stored for use in the next measurement, thereby maintaining measurement consistency.

In general, a measurement body part is a finger, and a means for measuring the pressure of a contact portion between the finger and an impedance electrode may be a pressure sensor, a blood sensor, a piezo sensor, a load cell, or an optical sensor. Any means capable of measuring a contact pressure may be used.

The principle of measuring a blood glucose level using the frequency-scan and impedance-measurement circuit of the present invention is shown in FIG. 9.

As variables, a function M for calculating a blood glucose level has: a multidimensional impedance value Z=[Z₁, Z₂, Z₃, . . . , Z_{N}]; and a weighting function W for adjusting the weight of each impedance measurement value.

Impedance values measured according to frequency are mapped to blood glucose levels using the function M. Here, variations in blood glucose having different sensitivities according to frequency are reflected in blood glucose measurement by using the weighting function W. The multidimensional impedance value Z is a multidimensional impedance value obtained for each resonant frequency band. In other words, a frequency band set for blood glucose measurement is divided into resonant frequency bands 1 to N, and while scanning frequency, the impedance of a circuit including the impedance electrode sensor is measured. Impedance values are calculated by multiplying impedance values measured as described above according to frequency by the weighting function W which is stored for reflecting sensitivity varying according to frequency.

This method is merely one of methods for measuring a blood glucose level using the impedance electrode sensor of the present invention, and the measurement method of the present invention is not limited thereto.

The weighting function may be determined by experiment or may be determined using a statistical, mathematical, or computer engineering method such as a multiple regression method or a deep learning method.

Solutions of the present invention for the above-described effects are as follows.

The present invention provides a non-invasive blood glucose sensor including: a measurement-unit body of the non-invasive blood glucose sensor;

an impedance electrode sensor provided on an inner bottom surface of the measurement-unit body;

a signal-generation and measurement unit configured to measure impedance while scanning frequency by supplying multiple frequencies to the impedance electrode sensor;

a pressure sensor configured to measure a contact pressure of a body part brought into contact with the impedance electrode sensor; and

a notification unit configured to indicate whether the contact pressure measured using the pressure sensor is appropriate.

In addition, the notification unit includes a status display LED configured to display green when the contact pressure measured using the pressure sensor is appropriate for non-invasive blood glucose measurement, yellow when the contact pressure is insufficient, and red when the contact pressure is excessive.

In addition, the impedance electrode sensor may be connected in parallel to a coil and in series to a capacitor to form a sensor circuit.

In addition, the impedance electrode sensor may be connected in series to a capacitor connected in parallel to a coil to form a sensor circuit.

As another means for solving the above-described problems, the present invention provides a non-invasive blood glucose sensor including:

a measurement-unit body of the non-invasive blood glucose sensor;

an impedance electrode sensor provided on an inner bottom surface of the measurement-unit body;

a signal-generation and measurement unit configured to measure impedance while scanning frequency by supplying multiple frequencies to the impedance electrode sensor;

a blood sensor configured to measure an amount of blood flowing through a blood vessel so as to measure a magnitude of a contact signal of a body part brought into contact with the impedance electrode sensor; and

a notification unit configured to indicate whether the amount of blood measured using the blood sensor is appropriate.

In addition, the notification unit includes a status display LED configured to display green when the amount of blood measured using the blood sensor is appropriate for non-invasive blood glucose measurement, yellow when the amount of blood is insufficient, and red when the amount of blood is excessive.

In addition, the impedance electrode sensor may be connected in parallel to a coil and in series to a capacitor to form a sensor circuit.

In addition, the impedance electrode sensor may be connected in series to a capacitor connected in parallel to a coil to form a sensor circuit.

1. A non-invasive blood glucose sensor comprising:
a measurement-unit body of the non-invasive blood glucose sensor;
an impedance electrode sensor provided on an inner bottom surface of the measurement-unit body;
a signal-generation and measurement unit configured to measure impedance while scanning frequency by supplying multiple frequencies to the impedance electrode sensor;
a pressure sensor configured to measure a contact pressure of a body part brought into contact with the impedance electrode sensor; and
a notification unit configured to display green when the contact pressure measured using the pressure sensor is appropriate for non-invasive blood glucose measurement, yellow when the contact pressure is insufficient, and red when the contact pressure is excessive,
wherein the impedance electrode sensor is connected in parallel to a coil and in series to a capacitor to form a sensor circuit for measuring a blood glucose level of a subject by using a phenomenon in which a tuning frequency decreases as the blood glucose level of the subject increases.

2. A non-invasive blood glucose sensor comprising:
a measurement-unit body of the non-invasive blood glucose sensor;
an impedance electrode sensor provided on an inner bottom surface of the measurement-unit body;
a signal-generation and measurement unit configured to measure impedance while scanning frequency by supplying multiple frequencies to the impedance electrode sensor;
a pressure sensor configured to measure a contact pressure of a body part brought into contact with the impedance electrode sensor; and
a notification unit configured to display green when the contact pressure measured using the pressure sensor is appropriate for non-invasive blood glucose measurement, yellow when the contact pressure is insufficient, and red when the contact pressure is excessive,
wherein the impedance electrode sensor is connected in series to a capacitor connected in parallel to a coil to form a sensor circuit for measuring a blood glucose level of a subject by using a phenomenon in which a Q value increases as the blood glucose level of the subject increases.

3. A non-invasive blood glucose sensor comprising:
a measurement-unit body of the non-invasive blood glucose sensor;
an impedance electrode sensor provided on an inner bottom surface of the measurement-unit body;
a signal-generation and measurement unit configured to measure impedance while scanning frequency by supplying multiple frequencies to the impedance electrode sensor;
a blood sensor configured to measure an amount of blood flowing through a blood vessel so as to correct a magnitude of a contact signal of a body part brought into contact with the impedance electrode sensor; and
a notification unit configured to indicate whether the amount of blood measured using the blood sensor is appropriate.

4. The non-invasive blood glucose sensor of claim 3, wherein the notification unit comprises a status display LED configured to display green when the amount of blood measured using the blood sensor is appropriate for non-invasive blood glucose measurement, yellow when the amount of blood is insufficient, and red when the amount of blood is excessive.

5. The non-invasive blood glucose sensor of claim 3, wherein the impedance electrode sensor is connected in parallel to a coil and in series to a capacitor to form a sensor circuit.

6. The non-invasive blood glucose sensor of claim 3, wherein the impedance electrode sensor is connected in series to a capacitor connected in parallel to a coil to form a sensor circuit.
